(19) United States
(12) Patent Application Publication
(10) Pub. No.: US 2022/0148679 A1
(43) Pub. Date: May 12, 2022

(54) Identification of Signature Mutations and Targeted Treatments

(71) Applicant: International Business Machines Corporation, Armonk, NY (US)

(72) Inventors: Claudia S. Huettner, Jamaica Plain, MA (US); Elinor Dehan, Haniel (IL); Bhuvan Sharma, Belmont, MA (US); Himanshu Sharma, Philadelphia, PA (US); Shang Xue, Santa Clara, CA (US)

(21) Appl. No.: 17/091,121

(22) Filed: Nov. 6, 2020

(51) Int. Cl. G16B 40/00; G16H 10/60; G16H 15/00; G16H 50/30; G06N 20/20

(52) U.S. Cl. CPC G16B 40/00; G16H 10/60; G16B 20/50; G16H 50/30; G06N 20/20; G16H 15/00

(57) ABSTRACT

A genomics artificial intelligence (AI) pipeline comprising a plurality of trained machine learning computer models is provided. First machine learning (ML) computer model(s) extract genomics entities from content of the electronic documents. Second ML computer model(s) determine relationships between genomics entities. Third ML computer model(s) grade biomarkers specified in the relationships based on a predetermined grading scheme and the relationships and gradings are stored in a genomics database for use in processing a patient gene sequencing data structure to identify a signature mutation. A report output is generated identifying the signature mutation present in the patient gene sequencing data structure.

### BACKGROUND

The present application relates generally to an improved data processing apparatus and method and more specifically to mechanisms for leveraging cognitive computing and artificial intelligence mechanisms to identify signature mutations and targeted treatments.

Mutational sequences are characteristic combinations of mutation types arising from specific mutagenesis processes, such as DNA replication infidelity, exogenous and endogenous genotoxins exposure, defective DNA repair pathways, and DNA enzymatic editing. Deciphering mutational sequences in cancer provides insight into the biological mechanisms involved in carcinogenesis and normal somatic mutagenesis. Advances in the fields of onco-genomics have enabled the development and use of molecularly targeted therapy. While molecularly targeted therapies identify mutations that may contribute to disease, they do so without clarifying or identifying the most critical mutation, i.e. the signature mutation, solely responsible for progression and metastasis. Nonetheless, mutational sequence profiling has proven successful in guiding oncological management and use of targeted therapies, e.g., immunotherapy in mismatch repair deficiency of diverse cancer types, and platinum and PARP inhibitor to exploit synthetic lethality in homologous recombination-deficient ovarian, breast, and prostate cancer.

### SUMMARY

This summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method is provided, in a data processing system comprising at least one processor and at least one memory, the at least one memory comprising instructions executed by the at least one processor to configure the at least one processor to implement a genomics artificial intelligence (AI) pipeline comprising a plurality of trained machine learning computer models. The method comprises processing, by at least one trained first machine learning (ML) computer model of the genomics AI pipeline, a corpus of electronic documents to extract genomics entities from content of the electronic documents. The method also comprises processing, by at least one trained second ML computer model of the genomics AI pipeline, the extracted genomics entities to generate one or more relationships between the extracted genomics entities. Moreover, the method comprises processing, by at least one trained third ML computer model of the genomics AI pipeline, the one or more relationships to grade biomarkers specified in the one or more relationships based on a predetermined grading scheme to thereby generate gradings for each of the one or more relationships. In addition, the method comprises storing, by the genomics AI pipeline, the one or more relationships in association with corresponding gradings of the one or more relationship in a genomics database. Furthermore, the method comprises processing, by a matching and consolidation module associated with the genomics AI pipeline, a patient gene sequencing data structure based on the genomics database to identify a signature mutation in the patient gene sequencing data structure by matching a gene mutation in the patient gene sequencing data structure to an entry in the genomics database corresponding to the signature mutation. In addition, the method comprises generating, by a report module associated with the genomics AI pipeline, a report output identifying the signature mutation present in the patient gene sequencing data structure. Thus, with these machine learning mechanisms, relationships between biomarkers, e.g., gene-mutations, and therapies may be learned and graded so as to identify signature mutations which can then be used to identify such signature mutations in patient gene sequencing data so as to report such to medical personnel for evaluation of therapies that can result in a best outcome for the patient.

In some illustrative embodiments, the at least one trained first ML computer model comprises a document classification ML computer model that is trained to classify electronic documents in the corpus of electronic documents as to types of clinical studies documented in content of the electronic documents, to thereby generate one or more subsets of electronic documents in the corpus of electronic documents, each subset corresponding to a different type of clinical study, and wherein the method further comprises executing the document classification ML computer model on electronic documents of the corpus of electronic documents and filtering out documents from further processing by the genomics AI pipeline, that have a predefined type. In this way, the genomics AI pipeline can eliminate electronic documents that likely do not provide useful genomics content for determining relationships between genomics entities from further processing via the AI pipeline, thereby saving computational resources and reducing errors that may be introduced by such documents.

In some illustrative embodiments, the at least one trained first ML computer model comprises a genomics entity extraction ML computer model that is configured, for each subset of electronic documents in the one or more subsets of electronic documents, to extract a subset of types of genomics entities based on a class of the subset of electronic documents. In this way, the genomics entity extraction ML computer model may be configured for different types of electronic documents based on the types of genomics entities that are typically present in content of those types of electronic documents and types of relationships documented by those types of electronic documents.

In some illustrative embodiments, the at least one trained second ML computer models comprise genomic relationship scoring logic that scores each relationship of the one or more relationships based on features specifying a clinical efficacy of a therapy associated with a genetic mutation specified in the relationship. In some illustrative embodiments, the scoring logic assigns different scores to different types of patient response to a corresponding therapy, and wherein, for each relationship in the one or more relationships, scores for instances in content of electronic documents of the corpus, of a gene mutation-therapy pair specified in the relationship, are accumulated across the instances to generate a score for the relationship, and wherein different therapies for a same gene mutation are ranked relative to each other based on corresponding accumulated scores for corresponding gene mutation-therapy pairs. The scoring of relationships based on features specifying a clinical efficacy of the therapy allows for relative ranking of therapies for the same genetic mutation so that the most effective therapy may be selected.

In some illustrative embodiments, processing the one or more relationships to grade biomarkers comprises, for each relationship in the one or more relationships, classifying, by the at least one trained third ML computer model, the relationship into a corresponding grade of the predetermined grading scheme, wherein the grading scheme comprises: a first grade indicating that no specific biomarker preclinical data or response is expected; a second grade indicating that biomarker responses have not been reported; a third grade indicating strong biomarker and strong response or lasting response in a plurality of patients; and a fourth grade indicating a signature mutation with complete remission or lasting partial response in patients. By grading the biomarkers, the mechanisms of the illustrative embodiments are able to identify signature mutations and thereby report the signature mutations to medical personnel for consideration of the associated therapy(s) for treating a patient since such signature mutations, when treated with the associated therapy(s), result in a complete remission or partial response of the patient.

In some illustrative embodiments, processing the patient gene sequencing data structure based on the genomics database to identify the signature mutation comprises: receiving, from a molecular profile analysis module, the gene sequencing data, wherein the gene sequencing data comprises driving gene-mutations present in a gene sequence for a tumor of the patient; receiving, from the molecular profile analysis module, an indicator of a type of cancer associated with the patient; performing a lookup operation in the genomics database a subset of entries corresponding to the type of cancer; and performing, by the matching and consolidation module, a lookup operation on the subset of entries corresponding to the cancer type, for each driving gene-mutation in the gene sequencing data, to find a corresponding matching entry in the subset of entries, if any. In this way, the patient gene sequencing data structure may be processed based on the particular type of cancer using the corresponding subset of entries rather than having to process the entire genomics database.

In some illustrative embodiments, generating the report output identifying the signature mutation present in the patient gene sequencing data structure further comprises accentuating the signature mutation in a display of the patient's genetic report and outputting a recommendation of a corresponding therapy based on the signature mutation, as indicated by the entry in the genomics database corresponding to the signature mutation. In this way, the attention of the medical personnel viewing the report output is brought to the signature mutation and corresponding therapy. As treatment based on the signature mutation and corresponding therapy will result in complete remission or partial response of the patient, accentuating its presence in the report and providing the recommended therapy improves the treatment of the patient, avoiding other therapies associated with non-signature mutations that are less effective.

In some illustrative embodiments, the genomics entities comprise gene mutations, therapies, medical conditions, and indicators of clinical efficacy of the therapies, and wherein the genomics entities are extracted from electronic documents of the corpus of electronic documents by executing natural language processing computer operations on content of the electronic documents. Thus, a specifically configured natural language processing is performed on electronic documents that is specific to genomics and the identification of signature mutations.

In some illustrative embodiments, the genomics entities and relationships between genomics entities are associated with one or more of solid tumors or hematology. Thus, the illustrative embodiments are able to performed the recited operations with regard to different types of cancers.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will be described in, or will become apparent to those of ordinary skill in the art in view of, the following detailed description of the example embodiments of the present invention.

### BRIEF DESCRIPTION OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 is an example diagram of a distributed data processing system in which aspects of the illustrative embodiments may be implemented;

FIG. 2A is an example diagram of signature mutations for solid tumors in accordance with one illustrative embodiment;

FIG. 2B is an example diagram of signature mutations for hematology cancers in accordance with one illustrative embodiment;

FIG. 3 is an example diagram of an example report that may be generated by a report module in accordance with one illustrative embodiment;

FIG. 4 is a flowchart outlining an example operation for automated extraction of gene-mutation relationships from a corpus of curated electronic documents, scoring therapies associated with the gene-mutation relationships, and grading gene-mutations in accordance with one illustrative embodiment;

FIG. 5 is a flowchart outlining an example operation for performing a report generation based on matching of driver gene-mutations in a patient's gene sequencing data with entries in a genomic database in accordance with one illustrative embodiment; and

FIG. 6 is an example block diagram of a computing device in which aspects of the illustrative embodiments may be implemented.

### DETAILED DESCRIPTION

Cancer is a malignant growth or tumor resulting from the division of abnormal cells caused by genetic alterations (referred to herein also as mutations or gene variants). With cancer, a diverse array of genetic lesions may be present, however some tumors rely on one single dominant mutation for growth and survival. With these types of tumors, inhibition of this specific alternation or mutation is sufficient to halt the neoplastic phenotype. This “signature mutation” is the “Achilles heel” of the tumor which can be exploited by successful molecular targeted therapies. Thus, the “signature mutation” is a mutation which is associated with the clinically most effective therapy for the medical condition, e.g., cancer type, in question. Targeted therapy of a signature mutation can induce either complete remission or long-lasting partial response with significant improvement of Quality of Life (QoL) for the patient.

However, it is often difficult to identify such signature mutations as the process to do so is still a manual process. This is especially difficult when one considers that a genetic case report for a tumor comprises all of the identified genetic alterations or mutations, the majority of which may be bystander mutations, i.e. non-signature mutations that do not play a major role in the disease, without pointing out the most relevant mutations (the terms “mutations”, “alterations”, and “gene variants” are used herein interchangeably to refer to genetic mutations that may be referenced in a genetic case report). That is, the genetic case report will state all pathogenic mutations that can be therapeutically addressed, resulting in an overload of information for a human oncologist traverse to identify pathogenic mutations that are likely to induce remission. The genetic case report thus represents a confusing variety of mutations that require intimate genetic knowledge and secondary reading by the human oncologist to go through in order to identify the most likely successful therapies for the patient. This may lead to important signature mutations not being properly identified within the genetic case report and thus, the failure to identify therapies that could drastically improve the patient's health.

With this in mind, the identification of a signature mutation within the genetic case report (or simply “case report”) represents the identification of the proverbial “needle in a haystack.” However, as the identification of such signature mutations can provide the identification of targeted therapies that can induce complete remission or long-lasting significant improvement in QoL, it is of critical importance that such signature mutations and associated therapy strategies be properly identified. Thus, it would be beneficial to avoid manual processes that represent essentially a brute force approach of manual review of a case report, fraught with sources of error, and provide more sophisticated automated computer tools to assist human beings in the treatment of cancer patients.

Illustrative embodiments of the present invention are directed to an improved computing tool that is specifically configured to implement automated signature mutation identification artificial intelligence (AI), referred to hereafter as a signature mutation AI system, which comprises executed AI computer models that approximate the results of a human thought process, but with computer AI specific processes that differ from human thought processes due to the inherent differences in the way that computer systems operate and the fact that computer systems do not have the ability to “think” the way that a human mind does. The signature mutation AI system of the illustrative embodiments do not involve any human mental processes and are not organizing any human activity, but rather are providing automated signature mutation AI mechanisms that operate without human intervention.

The signature mutation AI system provides automated computer tools to identify signature mutations and corresponding therapies that are applicable to a particular patient. The identification of the signature mutations and corresponding therapies is based on the patient's reported tumor profile, such as in a genetic case report for the patient, and learned signature genetic mutations. The signature mutation AI system identifies signature mutation therapies through learned associations of signature mutations and their therapies, as well as resistances to these therapies. The signature mutation AI system of the illustrative embodiments automatically applies machine learning based computer models and AI processes, without human intervention, to one or more corpora of electronic documents that document genetic knowledge, clinical studies, case reports, and the like, to automatically extract relationships between genes-mutations and therapies, and scores these relationships with regard to a degree of efficacy of the therapy on the medical condition given certain gene-mutations. From these scorings, signature mutations may be identified and the resulting extracted relations indicative of the signature mutations and related therapies for various genes-mutations may be stored in a genomic database for use by further AI computer models when evaluating an genetic case report of a patient. These further AI computer models may operate on a genetic case report for a patient to identify whether or not a signature mutation is present within the genetic case report for the patient.

In one illustrative embodiment, the signature mutation AI system comprises a computer executed AI pipeline of machine learning (ML) trained AI computer models that execute on a genetic case report generated by an automated computer tool that analyzes the biological markers (biomarkers)) obtained through sequencing of a patient's tumor DNA and generates a case report. The computer executed AI pipeline sets forth computer logic that identifies signature mutations based on the biomarkers such that automated AI processes, or executing of signature mutation rules, on the genetic case reports identifies one or more subsections of the genetic case report where biomarkers indicative of signature mutations may be present. Based on the identification of a signature mutation being present in the genetic case report, the signature mutation AI system identifies, from a genomic knowledge database, one or more corresponding therapies and, if present, biological resistance mechanisms that apply to the patient. This identification may be based on an execution of one or more ML based computer models and/or rules engines which are applied to a portion of the genomic knowledge database corresponding to the signature mutation to identify one or more therapies based on various features extracted from the genetic case report and/or patient electronic medical records (EMRs) including features associated with clinical efficacy, no efficacy, resistance, and pre-clinical efficacy of therapies specified in the genomic knowledge database.

Scoring of therapies may be performed based on the evaluation of these extracted features, as well as information extracted from knowledge resources indicative of the responsiveness of patients carrying the same biomarker to the therapies. Based on the scoring of the therapies, a therapy associated with the signature mutation is selected, e.g., the highest scoring therapy associated with the signature mutation, for recommendation to a medical practitioner for consideration-in treating the patient. The recommendation may identify the signature mutation and the corresponding recommended therapy. The recommendation may also identify other therapies that may be applicable to the signature and/or other mutations present in the patient's genetic case report (gene sequencing data structure), i.e. the patient's gene sequencing data.

Thus, the illustrative embodiments provide improved automated AI computer tools to generate a genetic knowledge database based on extraction of relations between genes-mutations and therapies specified in electronic documents, where these documents have content corresponding to genetic knowledge, clinical studies, case reports, etc., and a scoring of these relations with regard to degrees of efficacy on a medical condition given certain genes-mutations. In addition, the illustrative embodiments provide improved automated AI computer tools to identify signature mutations for a patient's medical condition as represented in a genetic case report for the patient. The illustrative embodiments further provide improved automated AI computer tools to score and rank therapies associated with a signature mutation so as to recommend therapies that are most likely to inhibit tumor growth and/or improve QoL of the patient. The illustrative embodiments further provide improved automated AI computer tools to present to a medical practitioner targeted information regarding recommended therapies for the signature mutation of the medical condition of the patient in a manner that highlights the signature mutation and presents clinical efficacy and resistance information to the medical practitioner for consideration when determining a therapy to treat the patient. It should be appreciated that these improved automated AI computer tools specifically provide computer tools that operate in a different manner than manual curation and specifically address the problems of manual curation that are inherent due to the overwhelming amount of information presented in genetic case reports and the overwhelming amount of information present in literature identifying genetic information and corresponding therapies.

That is, without recourse to the present invention, it is not possible for a human being to accurately determine therapies based on genetic case reports in a timely manner and with sufficient accuracy due to the immense amount of information present in genetic case reports. It would not be practical to perform such operations manually without significant possibility of error. Making this determination would involve manually searching through thousands of documents representing genetic information and corresponding therapies, each of which may contain only a morsel of specific, yet highly-relevant information, and then attempt to piece together the morsels from these thousands of documents to attempt to gain an understanding. For example, in the year 2018, the MEDLINE database contained references and abstracts on life sciences and biomedical topics which included more than 127,000 articles related to the oncology space. In the year 2019, this number rose to over 136,000. It is impossible for a human oncologist and/or pathologist to absorb the knowledge from this number of articles.

Moreover, one would need to then apply this acquired information to genetic case reports that may simply present all of the mutation information of the patient without any indicators of signature mutations and somehow determine the most clinically appropriate therapy. The inability to do this with any degree of practical efficiency often results in the human oncologist/pathologist having to rely on clinical guesswork without the benefit of full knowledge of the medical consequences of any decision. This guesswork is one of the principal causes of medical errors. Thus, the need for an artificial intelligence/cognitive computing tool, which can leverage the capabilities of such artificial intelligence/cognitive computing to achieve what a human being cannot with regard to specifically the ability of the artificial intelligence/cognitive computing system to identify signature mutations in genetic case reports and determine corresponding therapies is clear. The claimed invention is directed to such an improved artificial intelligence/cognitive computing system and is not directed to any manual process or mental process performed by a human being.

Before beginning the discussion of the various aspects of the illustrative embodiments and the improved computer operations performed by the illustrative embodiments, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on hardware to thereby configure the hardware to implement the specialized functionality of the present invention which the hardware would not otherwise be able to perform, software instructions stored on a medium such that the instructions are readily executable by hardware to thereby specifically configure the hardware to perform the recited functionality and specific computer operations described herein, a procedure or method for executing the functions, or a combination of any of the above.

The present description and claims may make use of the terms “a”, “at least one of”, and “one or more of” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine,” if used herein with regard to describing embodiments and features of the invention, is not intended to be limiting of any particular implementation for accomplishing and/or performing the actions, steps, processes, etc., attributable to and/or performed by the engine. An engine may be, but is not limited to, software, hardware and/or firmware or any combination thereof that performs the specified functions including, but not limited to, any use of a general and/or specialized processor in combination with appropriate software loaded or stored in a machine readable memory and executed by the processor. Further, any name associated with a particular engine is, unless otherwise specified, for purposes of convenience of reference and not intended to be limiting to a specific implementation. Additionally, any functionality attributed to an engine may be equally performed by multiple engines, incorporated into and/or combined with the functionality of another engine of the same or different type, or distributed across one or more engines of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

The illustrative embodiments provide an automated AI pipeline that comprises one or more machine learning (ML) trained computer models that are specifically trained through ML processes to identify patterns of input features that are indicative of particular classifications or outputs of the ML trained computer models. For example, the ML trained computer models may be trained based on ground truth inputs, e.g., collections of input data comprising input features which are manually annotated or otherwise associated with correct classifications/outputs that the ML trained computer models should generate when properly trained. The computer model processes the ground truth inputs to generate a prediction of an output/classification which is then compared to the correct classification/output to identify a loss or error in the operation of the computer model. This loss or error is then used by ML training logic to determine an adjustment in operational parameters, e.g., weights of nodes, of the computer model to reduce the loss or error. The process is repeated through multiple epochs until an acceptable level of loss/error is achieved, or a predetermined number of epochs are performed. With each epoch, the operational parameters of the computer model are adjusted to attempt to reduce the loss/error such that convergence of the computer model is preferably achieved. At this point, the computer model is determined to have been trained and thus, represents a trained ML computer model.

It should be appreciated that such ML computer models may take various different currently known or later developed forms, such as convolutional neural networks, recurrent neural network computer models, deep learning neural network computer models, decision tree computer models, support vector machine (SVM) computer models, and the like. These ML computer models may be trained to perform various types of tasks. For example, ML computer models may include classification models, regression models, clustering models, dimensionality reduction, deep learning, and the like.

Classification ML computer models attempt to predict a type or class of an object within a finite number of options and may generate binary classification outputs or multiple classification outputs, e.g., output vectors in which each vector slot represents a different class and the value in each vector slot represents a probability that the input is properly classified in the associated class. Examples of ML computer models that are of the classification model type include, but are not limited to, K-nearest neighbor computer models, naïve Bayes computer models, logistic regression computer models, SVM computer models, decision tree computer models, and the like.

Regression ML computer models attempt to predict the value of a continuous variable by finding the relationship between a dependent variable and one or more independent variables. Examples of regression models include, but are not limited to, linear regression models, Lasso regression models, ridge regression models, SVM regression models, and decision tree regression models.

Clustering ML computer models operate to group similar objects together without manual intervention. Examples of clustering ML computer models include, but are not limited to, K-means computer models, K-means++ computer models, Agglomerative clustering computer models, density-based clustering algorithm computer models, and the like.

Dimensionality reduction ML computer models utilize the concept of “dimensionality” which is the number of predictor variables used to predict an independent variable or target. In real-world datasets, the number of variables is very large and too many variables lead to overfitting the model to the data. Moreover, not all of the variables contribute equally towards the desired operation of the computer model. Thus, the dimensionality reduction ML computer models operate to reduce the dimensionality of computer models by performing embedding of higher-dimensional data into lower dimensional representations. Examples of such dimensionality reduction ML computer models include principle component analysis (PCA), t-distributed stochastic neighbor embedding (t-SNE), singular value decomposition (SVD), and the like.

Deep learning ML computer models are a subset of machine learning that uses multiple layers to progressively extract higher-level features from raw input data. For example, in image processing, lower layers may identify edges, while higher layers may identify the concepts relevant to a human, such as digits, letters, or faces. Deep learning ML computer models use neural networks, such as multi-layer perceptron, convolutional neural networks (CNNs), recurrent neural networks (RNNs), Boltzmann machines, autoencoders, and the like.

The machine learning may be performed using any known or later developed machine learning logic, algorithms, and techniques. For example, the machine learning may be performed using supervised learning, unsupervised learning, reinforced learning, or the like. With supervised learning, the goal is to predict a target or an outcome variable from a set of independent variables. A function map is generated that maps inputs to a desired output with this function map being iteratively modified until a desired accuracy is achieved. Examples of supervised learning trained ML computer models include decision tree, K-nearest neighbor (KNN), regression, and logistic regression models. Unsupervised learning is a type of machine learning that looks for previously undetected patterns in a data set with no pre-existing annotations (labels) and with minimum human supervision. Unsupervised learning models probability densities over inputs. Reinforced learning is a type of machine learning concerned with how to take actions in order to maximize a cumulative reward by locating an ideal method to achieve a specific objective. In such reinforced learning, as an operator makes a move that goes toward an objective, the operator gets a reward and anticipates the best following stage to procure the most reward.

The illustrative embodiments provide an automated artificial intelligence (AI) pipeline comprising a plurality of trained ML computer models that are each trained to perform respective operations such as document classification, entity extraction, relationship extraction, therapy scoring, gene-mutation grading, and the like. The ML computer models may be configured to be a corresponding one or more of the above types of ML computer models. For example, the document classification ML computer model, as well as the gene-mutation grading ML computer model, may be a classification ML computer model. In some cases, while a single ML model is depicted, the single ML model may in fact be implemented as an ensemble of ML models trained to work together to perform a corresponding function.

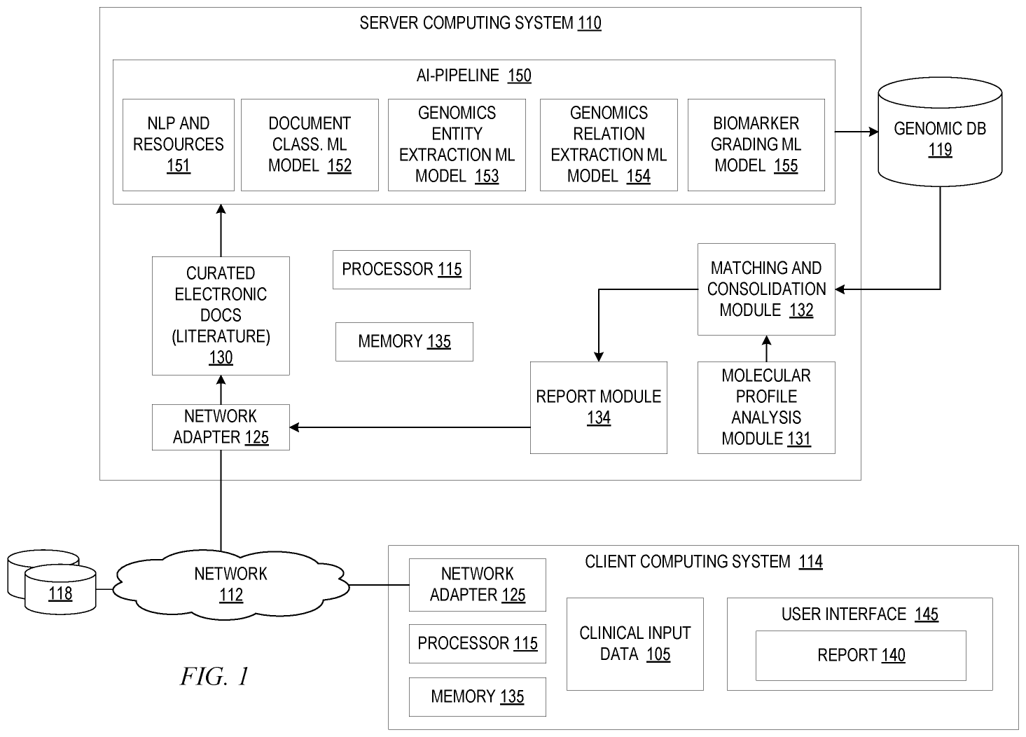

An example environment 100 for use with present invention embodiments is illustrated in FIG. 1. The example environment includes one or more server systems 110, and one or more client or end-user systems 114. Server systems 110 and client systems 114 may be remote from each other and communicate over a network 112. The network may be implemented by any number of any suitable communications media (e.g., wide area network (WAN), local area network (LAN), Internet, Intranet, etc.). Alternatively, server systems 110 and client systems 114 may be local to each other, and communicate via any appropriate local communication medium (e.g., local area network (LAN), hardwire, wireless link, Intranet, etc.).

Client systems 114 enable users to view reports (e.g., case summaries, genes, gene variants, variant types, condition names, evidence, prognoses, diagnoses/diseases, cancer types, predispositions, mutations (e.g., somatic or germline), treatments, etc.) from server systems 110. The server systems include various modules for analyzing and consolidating information as described herein. Moreover, the server systems, in accordance with one or more illustrative embodiments, provide an artificial intelligence (AI) pipeline comprised of a plurality of machine learning (ML) computer models that are trained to perform various AI operations with regard to identifying relationships between genetic mutations and therapies in a vast collection of electronic documents and scoring such relations with regard to efficacy on a medical condition given a gene-mutation. The AI pipeline further comprises one or more ML computer models specifically trained to process genetic case reports for patients and generate reports highlighting signature mutations and corresponding therapies.

With the mechanisms of the illustrative embodiments, a corpus or corpora 118 may provide data for analysis that is stored as curated literature, and the genomic database 119 may store information, including extracted relationships between genetic mutations, therapies, classifications of genetic mutations and therapies with regard to efficacy for one or more medical conditions, e.g., cancers or other genetic diseases, and grading of mutations/therapies, obtained through an AI pipeline 150 based ingestion and analysis of the curated literature in the corpus/corpora 118. The curated literature may be “curated” in that the literature (electronic documents) included in the corpus/corpora 118 may specifically selected from particular sources known to provide quality content for a particular domain, e.g., genomics in the illustrative embodiments. Thus, the corpus/corpora 118 may be a collection of electronic documents obtained from selected sources, e.g., medical journals, clinical studies, etc. In some aspects, the curated literature of the corpus/corpora 118 may comprise structured information. In other aspects, the curated literature of the corpus/corpora 118 may include natural language electronic documents, which may be manually created/reviewed and annotated by subject matter experts (SMEs), where such annotations may tag specifically identify instances of types of information present in the electronic document, and specifically in the context of genomics for purposes of the illustrative embodiments.

The AI pipeline 150 may comprise natural language processing (NLP) computer logic and corresponding resource data structures 151 for parsing structured content and/or unstructured natural language content of the electronic documents in the corpus/corpora 118, and identifying language features of the natural language content in the structured/unstructured content based on various data resources including dictionaries, synonym data structures, ontologies, and the like. The natural language processing may be used to extract features from the structured/unstructured natural language content and provide those extracted features as inputs to one or more of the trained machine learning (ML) computer models of the AI pipeline 150 so as to perform classification, entity extraction, relation extraction, grading of mutations/therapies, and identification of signature mutations/therapies based on the gradings. The NLP computer logic and corresponding resources 151, in accordance with the illustrative embodiments, are specifically configured for performing NLP operations with regard to genomics content. For example, the NLP computer logic is configured to parse and extract features specifically associated with genetic medical conditions, genes, mutations, therapies (e.g., drugs and the like), indicators of clinical efficacy, and the like. In some illustrative embodiments, this specific configuration comprises using specific provided dictionaries, ontologies, listings of recognized entities, and the like, that are tailored to the particular domain of interest, e.g., genomics.

The result of the processing of the electronic documents of the corpus/corpora 118 through the AI pipeline 150 is a genomic database 119 that identifies, in addition to other genomic information, the grading of relationships between mutations and therapies as determined from the extracted relationships in the various documents, and the identification of signature mutations based on these gradings. In some illustrative embodiments, the genomics database 119 may be initially populated with a seed set of genomics knowledge with regard to particular genes and gene mutations. For example, these resource data structures may include a set of seed genomics database information specifying known genes, gene-mutations, and classifications and gradings of these gene-mutations with regard to clinical efficacy in accordance with the grading scheme of the illustrative embodiments. This initial seed set of genomics database information may be automatically expanded through the operation of the AI pipeline 150 as described herein.

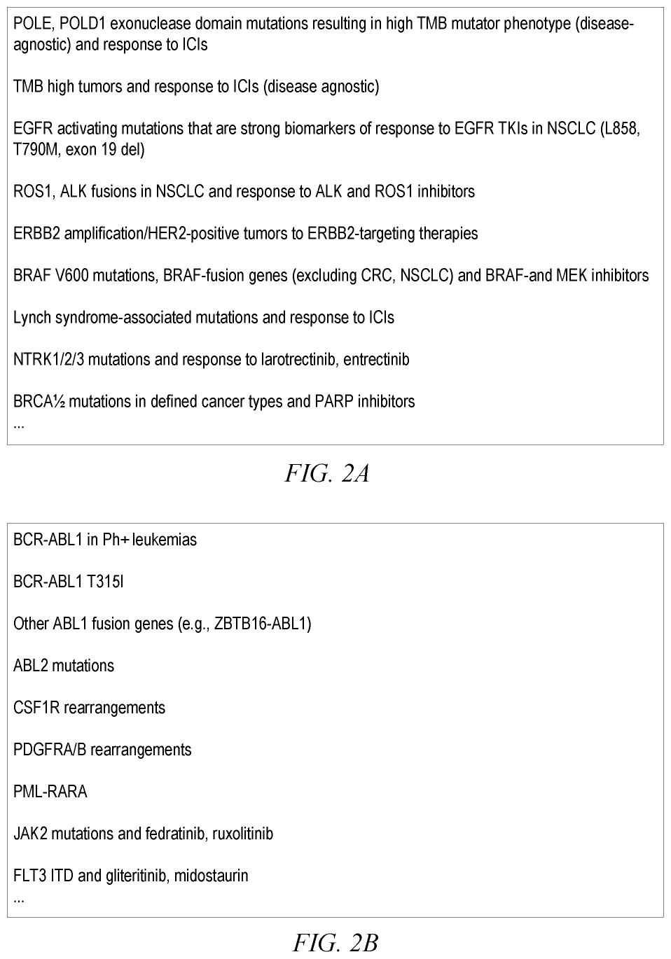

For example, the seed genomics database information may specify known signature mutations. These signature mutations may be specified with regard to different types of medical condition, e.g., solid tumors, hematology, etc. FIGS. 2A and 2B show examples of signature mutations for solid tumors and hematology, respectively. These signature mutations may be provided by subject matter experts as part of the seed genomics database information and/or may be learned through machine learning processes of the illustrative embodiments as described hereafter. The automated or semi-automated machine learning process includes the extraction of clinical information using natural language processing and machine learning algorithms which learn relationships between recognized features extracted from the published literature. For example, the natural language processing (NLP) mechanisms may be specifically configured to identify features specific to gene variants (also referred to as alterations or mutations), specific drugs or therapies, and clinical responses to such drugs or therapies. The machine learning computer model(s) are trained through machine learning processes and learn relationships between these extracted features, e.g., relationships between gene variants, specific drugs or therapies, and clinical responses. Mutations that have been identified in the published literature as driver mutations that are solely responsible for progression of disease, where targeted inhibition resulted in either complete remission or lasting stable disease (no progression) in patients, are classified as signature mutations. Thus, the machine learning (ML) mechanisms of the illustrative embodiments learn signature mutations and their corresponding therapies which can then be used to expand the genomics database information and can be used to identify the presence of such signature mutations in other patients and provide recommendations as to applicable therapies.

Thus, with the mechanisms of the illustrative embodiments, seed signature mutations specified in the seed genomics database information may be expanded by automated or semi-automated machine learning and graded through the AI pipeline 150 operations as described herein, such that the genomics database 119 may be dynamically updated as new documentation is added to the corpus/corpora 118. In some illustrative embodiments, periodic processing of the corpus/corpora 118, or updated portions of the corpus/corpora 118, may be performed by the AI pipeline 150 such that the grading of gene-mutations and their corresponding therapies may be kept current and accurate with regard to clinical studies, genomics specific publications, and the like.

Thereafter, the matching and consolidation module 132 may utilize the genomic database 119 to evaluate genetic case reports for patients with regard to these relationships, gradings, and importantly the signature mutations. The matching and consolidation module 132 may perform a rules based matching with the genomic database 119, or may implement a trained machine learning computer model to perform its matching and evaluation of the patient's genetic case report to identify therapies applicable to the patient and to highlight instances of signature mutations and their corresponding therapies, as described hereafter.

As shown in FIG. 1, the AI pipeline 150 comprises a plurality of trained ML computer models 152-155 in addition to the NLP logic and resources 151. A first ML computer model 152, referred to as the genomics documentation classification ML computer model 152, of the AI pipeline 150 is specifically trained, through machine learning processes, to perform classification of electronic documents in the corpus/corpora 118 with regard to clinical, functional, and resistance studies in the genomics domain. The genomics documentation classification ML computer model 152 may be trained on a training set of documents that are manually annotated (labeled) with proper classifications with regard to each of a plurality of predefined classes. In one illustrative embodiment, the classes may include, for example, types of studies documented in the corresponding document. For example, the classes of studies may include study types of “Functional”, “Clinical”, “Predisposition”, “Preclinical”, “Resistance”, and “Other”. In one illustrative embodiment, any documents categorized as “Other” are ignored while the rest are processed by downstream NLP components.

One differentiating feature between the above classes is the kind of entity associations discussed in the natural language content of the document and thus, the types of features and associations between features that may be extracted from the natural language content of the documents. A “Functional” study discusses association between a gene and a variant or fusion gene (which is also a kind of variant). A “Predisposition” study discusses associations between a gene, variant (alteration or mutation), and medical condition. A “Resistance” study discusses associations between gene, variant and therapy. A “Preclinical” or “Clinical” study discusses associations between gene, variant, therapy (e.g., drugs), and medical condition, with the “Preclinical” study documentation focusing on results on cell lines (lab settings), while the “Clinical” study documentation focuses on results in actual patients. The purpose of doing this classification is to ensure that downstream NLP and machine learning operations focus on extraction of only relevant entities and features depending on the particular study type. For example, if a document has content describing a “Functional” study, the NLP entity detection and relation extraction components are configured to focus on extracting gene and variant mentions in the natural language content of the document only, and any relations expressed between the gene-variant tuple. In some illustrative embodiments, results from certain types, or classes, of studies (clinical and resistance for example) may be given higher importance, or weighting, in the operation of the mechanisms of the illustrative embodiments with regard to inferring signature mutations, based on the desired implementation.

The genomics documentation classification ML computer model 152 operates to parse the content of an input electronic document, with the aid of the NLP logic and resources 151, and converts the content into a vector representation of dimension equal to the number of unique tokens in the content. A value is assigned to each dimension equal to the frequency of the corresponding token. The frequency pattern of all the tokens in the training set of documents is used for training the genomics documentation classification ML computer model 152 to learn the distribution of frequency values for tokens in each class. After learning, for each class, the distribution of frequency values for tokens corresponding to that class, when a new electronic document is received, the frequency distribution of its tokens is compared with the learned frequency distributions for each of the classes and the new electronic document is then assigned to a class having a closest matching frequency distribution. As noted above, the particular entity/feature extraction and relationship extraction performed by further downstream models of the AI pipeline 150 may be configured based on the classification of the documents being processed, so as to extract the types of entities/features and relationships that are present in the natural language content of the documents of that particular type/class.

That is, the AI pipeline 150 further includes a genomics entity extraction ML computer model 153 which is trained to operate on electronic documents, with the aid of the NLP logic and resources 151, to parse the content of the electronic documents and identify key entities, e.g., genes, drugs, therapies, mutations, medical conditions, etc., mentioned in the content. The NLP logic and resources 151 may be used to configure the genomics entity extraction ML computer model 153 based on the particular class of the documents being processed as indicated by the document classification ML model 152. The entity extraction comprises a preprocessing of the electronic document to generate a vector representation of the content of the electronic document, e.g., using a word2vec, doc2vec, GloVe, or other tool that generates a dense embedding of the content, which is then fed as input into a deep learning neural network based computer model that generates an output of recognized entities referenced in the content. Such deep learning neural network entity recognition mechanisms are generally known in the art, however, these mechanisms are specifically configured by the mechanisms of the illustrative embodiments to with regard to entities/features specific to genomics. Moreover, these entity recognition mechanisms are specifically configured by the mechanisms of the illustrative embodiments to extract entities/features from documents based on the particular classification of the documents being processed, as discussed above. Thus, even though the underlying entity extraction algorithms themselves may be known, the particular configuration of these underlying algorithms specifically based on the classification of the documents being processed and specifically with regard to genomics entities, such as gene, variant, therapy, response, etc., is not known in these general entity extraction algorithms.

The AI pipeline 150 further includes a genomics relation extraction ML computer model 154 which is trained through machine learning processes to generate hypotheses about the interaction of a therapy and a gene-mutation, given a medical condition. The genomics relation extraction ML computer model 154 generates hypotheses as to whether a particular therapy, e.g., one or more drugs, mentioned in content of an electronic document has some efficacy with regard to the given medical condition given a gene-mutation, e.g., clinical efficacy, no efficacy, resistance, pre-clinical efficacy. While the genomics relation extraction ML computer model 154 is shown as a single ML computer model, in some illustrative embodiments, this ML computer model 154 may in fact be an ensemble ML computer model including kNN computer models, SVM computer models, logistic regression computer models, and/or the like. The genomics relation extraction ML computer model 154, based on the entity extraction performed by the genomics entity extraction ML computer model 153, identifies relationships between these entities specified in the content of the electronic documents, e.g., medical conditions, corresponding gene-mutations, corresponding therapies (e.g., one or more drugs), corresponding indicators of clinical efficacy, and the like.

These hypotheses are generated by evaluating the language in the content of the electronic document to determine the semantics of terms used in the content with regard to the extracted entities. For example, if a document states, in the context of extracted entities corresponding to a particular gene-mutation and therapy, that the patient experienced “complete remission” (CR), then the relationship of gene-mutation/therapy/CR may be generated based on this content. As another example, if the content of the electronic document indicates a “partial response” (PR) by the patient to a specified therapy and that the patient had a particular gene-mutation, then again a relationship of gene-mutation/therapy/PR may be generated. In yet another example, if the content of the electronic document indicates a “stable disease” (SD) with regard to a therapy, and the patient has a particular gene-mutation, then again the relationship of gene-mutation/therapy/SD may be generated. These relationships may be defined and stored as tuple data structures, such as {gene-mutation, therapy, patient response}, for example.

In the examples of patient response above, the possible response types or classifications are complete remission (CR), partial response (PR), and stable disease (SD). These are provided as examples and the illustrative embodiments are not limited to these classifications of patient response. In these examples, a complete remission (CR) response represents that tests, physical exams, and scans show that there are no signs of cancer. A partial response (PR) indicates that the cancer partly responded to the therapy, but the tumor is still present, where a partial response (PR) is most often defined as at least a 50% reduction in measurable tumor. A stable disease (SD) response indicates that the therapy prevented the tumor from growing but it was not able to result in a reduction in the tumor, i.e. there is neither an increase in size of more than X % nor a decrease in size of more than Y % since an initial baseline measurement, where X % and Y % may be selected based on the desired implementation, e.g., X=20% and Y=30%. Those of ordinary skill in the art, in view of the present description, will recognize that these are only examples and other types of patient response or classifications of patient response to therapies may be used without departing from the spirit and scope of the present invention.

It should be appreciated that the natural language terms or phrases, or other types of entities, such as medical codes, numerical values, and the like, (collectively referred to herein as “terms”) for determining the patient's response to a therapy may be varied and may not specifically indicate the particular class of response, e.g., CR, PR, SD, or the like. The terms may be evaluated by the genomics relation extraction ML computer model 154 to determine probabilities of particular patient response classifications for the relationship. For example, if a statement in the content of an electronic document indicates that a patient with gene-mutation A, given therapy B, had a significant reduction in symptoms, or the content indicates a particular percentage reduction in numbers of tumors present, or the like, this evidence may be evaluated by the genomics relation extraction ML computer model to generate a probability value for one or more clinical efficacy classifications, e.g., complete remission (CR), partial response (PR), stable disease (SD), no response (NR), etc. In this case, terms such as “significant” may be indicative of a “partial response” and thus, the probability associated with this classification may be increased, whereas certain percentage reductions may be evaluated to determine if they are indicative of a partial response or a stable disease, or no response. All of the evidence in the electronic document may be evaluated with regard to the extracted entities and features of gene-mutation/therapy specified in the electronic document, and corresponding patient response class probability values may be generated so as to determine an appropriate relationship between gene-mutation, therapy, and patient response.

It should be appreciated that the entity/feature extraction and identification of relationships between entities/features may be performed across multiple documents of the same or different classes, which may together be part of a corpus of documents or corpora of documents. Thus, for example, one document may specify part of the relationship between gene-mutation and therapy, while another document may specify relationships between therapies and patient response. Moreover, various documents may specify different relationships for the same entities, e.g., different patient response to the same therapy, different gene-mutations and therapies, etc. Moreover, the processing of the documents of the one or more corpora may be performed over time such that the relationships are maintained up to date. While each document may be processed individually, the results of such processing over multiple documents and over time is joined by the extracted entities/features and relations. For example, if an extracted entity is the BRAF gene and V600E mutation, instances of these entities obtained across multiple documents containing content referencing relations express between these entities is combined through the mechanisms of the illustrative embodiments. Thus, for example, sentences such as “A patient with BRAF(L597S) mutant metastatic melanoma responded significantly to treatment with the MEK inhibitor, TAK-733” and the sentence “As mentioned previously, similar to cabozantnib, an overall response rated of 33% can be achieved with the use of single-agent BRAF inhibition (dabrafenib) in BRAF V600E-mutant lung cancers” in the same or different document may be used to extract entities/features and determine relationships between gene-mutation, therapy, and patient response using the mechanisms of the illustrative embodiments, e.g., {BRAF V600E, TAK-733, PR} and {BRAF V600E, dabrafenib, PR}.

In some illustrative embodiments, the machine learning training of the genomics relation extraction ML computer model 154 may implement a “distant supervision” machine learning process. The “distant supervision” machine learning process differs from other machine learning processes, such as supervised or unsupervised machine learning, in that distant supervision is a combination of automatically generated training data and supervised learning. Usually, in supervised learning, training data is manually generated by humans doing the labeling of the training data based on their own expertise. In distant supervision, this task is automated by looking at a known knowledge bank, and inferring ground truth using that known knowledge bank.

In the context of the illustrative embodiments, the known knowledge bank may be a seed set of genomics knowledge with regard to particular genes, gene mutations, therapies, etc., and relationships between these entities. Based on this seed set of genomics knowledge, electronic documents of a training corpus may be processed to identify instances of these entities and relationships and automatically label them in the electronic documents of the training corpus. The automatically labeled training data may then be used to train the genomics relation extraction ML computer model 154, using a supervised machine learning process, such that the trained genomics relation extraction ML computer model 154 is able to identify mentions of relationships between genomics entities that are outside those specified in the seed set of genomics knowledge.

The genomics relation extraction ML computer model 154 may further include a relationship scoring component that scores each of the relationships, e.g., {gene-mutation, therapy, patient response}, with regard to patient response to the therapy. For example, each gene-mutation may have a corresponding set of therapies specified in the electronic documents of the corpus/corpora 118. For each therapy of each gene-mutation, the patient response is determined to be one of a plurality of pre-defined patient response classifications, e.g., complete remission (CR) meaning that the patient is disease-free (cancer-free in the case of cancer), partial response (PR) indicating a significant reduction in the number of tumors, stable disease (SD) indicating that the disease does not progress for a measurable amount of time, and no response (NR) indicating that the patient did not respond to the therapy. Each of these patient response classifications may have a corresponding point value or score associated with it. For example, in one illustrative embodiment, CR may be given 1 point, PR may be given 3 points, SD may be given 5 points, and NR may be given 10 points. These points may be accumulated for each pairing of gene-mutation and therapy across multiple relationships identified in one or more electronic documents of the corpus/corpora 118.

In this illustrative embodiment, after accumulating the scores across these relationships, the genomics relation extraction ML computer model 154 identifies the most relevant therapy for a particular gene-mutation (biomarker) as the therapy having the lowest accumulated score. It should be appreciated that the relationships and their associated scores may be stored in the genomics database 119 along with other genomic information for the gene-mutation and therapy, for later use, such as by the matching and consolidation module 132.

It should also be appreciated that this is only one example of a scoring mechanism that may be used to score relationships and other scoring or ranking schemes may be used without departing from the spirit and scope of the present invention. For example, rather than using the specific point assignments noted above, other point assignments may be utilized. Moreover, rather than looking for the lowest scoring therapy, in other illustrative embodiments, depending on the scoring scheme utilized, a greatest scoring therapy may be selected as the most relevant therapy for a particular gene-mutation (biomarker).

Having identified relationships between gene-mutations, therapies, and patient responses, and having scored the therapies for each of the gene-mutations/therapy pairs so as to identify the most relevant therapy for a particular gene-mutation, which is also called a biomarker of response to a certain therapy, the mechanisms of the illustrative embodiments further provide the biomarker grading ML computer model 155 that applies a grading scheme to grade each gene-mutation based on the evaluation of patient responses to the most relevant therapies, such as determined by the scoring mechanisms of the genomics relation extraction ML computer model 154.

The biomarker grading ML computer model 155 may be implemented as a trained ML computer model performing a classification operation where the classifications are the defined gradings of the grading scheme. The biomarker grading ML computer model 155 is trained, through a machine learning process, to grade gene-mutations as to patient responses across multiple patients. That is, having identified pairings of gene-mutations with therapies, and identified the most relevant therapies through the patient response scoring mechanism described above, the biomarker grading ML computer model 155 may process a subset of the corpus/corpora 118 whose content specifically references the gene-mutation and most relevant therapy. This may identify multiple electronic documents documenting patient responses to the relevant therapy. The vector representation of these electronic documents, e.g., the frequency of occurrence of tokens and the like as previously described above, may be input to the biomarker grading ML computer model 155 which then evaluates these input features to generate a prediction as to a grading of the gene-mutation with regard to the predefined grading scheme. This prediction may then be used to update the corresponding entry in the genomic database 119 with the grading of the gene-mutation.

The biomarker grading ML computer model 155 uses a grading scheme comprising a plurality of predefined grades for biomarkers based on patient responses to the most relevant therapy associated with that biomarker. In one illustrative embodiment, the grading scheme comprises a scale of four grades defined as follows:
Grade IIII—Signature mutation with complete remission (CR) for preferred treatment or lasting partial response (PR) in advanced disease in all patients;
Grade III—Strong biomarker and strong response or lasting response in many patients;
Grade II—biomarker responses have not been reported; and
Grade I—No specific biomarker association preclinical data or response is expected.

Examples of Grade IIII biomarkers include BRAF V600E/K except for colorectal cancer (CRC), lung cancer; EGFR L858R, T790M; and JAK2 V617 F. These are well-characterized mutations known to be sufficient to cause cancer, they are the key drivers of the disease. Blocking them will result in complete remission in all patients or lasting stable disease in patients with a large tumor burden. Examples of Grade III biomarkers include KIAA1549-BRAF and EGFR G719A. These are mutations that are also well-defined and sufficient to cause cancer, but they are relatively rare compared to Grade IIII, i.e. the signature mutations. They are not explicitly stated by FDA approvals but efficacy has been demonstrated in many clinical reports. Examples of Grade II biomarkers include PIK3CA mutations in Her2+ breast cancer and ERBB2 S310Y in NSCLC. Examples of Grade I biomarkers include TSC2 mutations and everolimus in any cancer type.

It should be appreciated that this is only one example of a grading scheme that may be utilized to grade biomarkers with regard to patient response to the most relevant therapies. Other grading schemes may be used without departing from the spirit and scope of the present invention, with these grading schemes having more or fewer grades that those in this illustrative example and grades that may be defined differently from those described above.

Based on the grading scheme implemented, and assuming for illustrative purposes the grading scheme mentioned above, the biomarker grading ML computer model 155 is trained to classify gene-mutations into one or more of these grades. For example, given information about BRAF V600E/K and its most relevant therapy, the electronic documents of the corpus/corpora 118 that reference BRAF V600E/K and the relevant therapy are identified from the corpus/corpora 118 and their encoded vector representations are input to the biomarker grading ML computer model 155 which generates a prediction of a corresponding grade I-IIII by generating probability values for each grade I-IIII and selecting a highest probability grading as the final grading for the input. In this example, the output vector of the biomarker grading ML computer model 155 may be a four slot vector output where each vector slot represents a separate one of the grades I-IIII. During training, the output vector may be compared to a ground truth grading of the gene-mutation to determine an error or loss and appropriate machine learning logic based adjustment of operational parameters of the biomarker grading ML computer model 155 are performed to attempt to reduce the error or loss, with this process being repeated through multiple epochs until convergence (acceptable error/loss is achieved) or a predetermined number of epochs is reached. During runtime operation, the output vector is used to assign a grading to the gene-mutation and corresponding most relevant therapy. This grading is then stored in association with the entry in the genomic database 119 corresponding to the gene-mutation and relevant therapy.

Thus, through the operation of the AI pipeline 150, relationships between gene-mutations, therapies, and patient responses may be automatically identified through machine learning based AI processing of a corpus/corpora 118 of domain specific electronic documents (curated literature). These relationships may be scored according to a therapy scoring scheme to identify the most relevant therapies for the gene-mutations, where the most relevant therapies are those that provide the best patient responses, e.g., complete response, partial response, etc. The identification of the most relevant therapy for a gene-mutation may then be used to select a subset of the corpus/corpora 118 that references the most relevant therapy for the gene-mutation and evaluate the subset with regard to a grading scheme for grading gene-mutations with regard to patient responses to relevant therapies. The grading scheme identifies signature mutations which are indicative of mutations that if treated with a relevant therapy, will result in complete response, i.e. induce either complete remission or long-lasting partial response with significant improvement of Quality of Life (QoL) for the patient. For example, gene-mutations that are graded by the biomarker grading ML computer model 155 as grade IIII in the example grading scheme noted above, are considered signature mutations such that if a patient has this gene-mutation in their genetic case report, a corresponding most relevant therapy for that gene-mutation will most likely cause a complete response in the patient.

From the AI pipeline 150 based processing of the corpus/corpora 118, the genomic database 119 is built with entries for each of the gene-mutations represented in the content of the electronic documents of the corpus/corpora 118. The genomic database 119 may contain tables and/or other data structures which the matching and consolidation module 132 uses for determining a prognosis, a diagnosis, and/or a predisposition, as well as identify the most relevant therapies based on the grading of gene-mutations and scoring of therapies associated with these gene-mutations. In some aspects, the genomic database 119 may contain gene names, variant names, variant type information, condition names, evidence, summary information, prognostic information, predisposition information, and diagnostic information. In accordance with the illustrative embodiments, the genomic database 119 may further include information identifying gradings of gene-mutations, most relevant therapy, and the like. In some aspects, this information may be provided in a structured format.

Matching and consolidation module 132 may consolidate various types of information for a genetic report output (e.g., prognosis, diagnosis, predisposition information, grading, relevant therapy, etc.). Matching and consolidation module 132 along with input from molecular profile analysis module 131 may perform hierarchical matching of gene-mutations specified in a molecular profile (genetic case report) for a given patient as identified by the molecular profile analysis module 131 to entries corresponding to the gene-mutations represented in the genomic database 119. That is, a client system 114 may represent a computing device at a medical facility that performs gene sequencing and generates gene sequencing data/cancer type information, as well as gathers other clinical input data 105, which is provide to the molecular profile analysis module 131. The molecular profile analysis module 131 identifies the driving gene-mutations present in the gene sequencing data and provides the driver gene-mutation and cancer type (medical condition) information to the matching and consolidation module 132. The matching and consolidation module 132 then identifies the entries in the genomic database 119 corresponding to the cancer type (medical condition) and performs a matching operation of the gene-mutations with the entries in the genomic database 119 corresponding to the cancer type. The resulting genomic information from the genomic database 119 for matching entries may then be returned for use in generating a genetic report by the report module 134.

Of particular importance, the matching and consolidation module 132 may identify matching entries in the genomic database 119 that corresponding to signature mutations, e.g., gene-mutations with gradings of IIII in the example grading scheme discussed above. For those matching gene-mutations that match signature mutations, these entries are highlighted or otherwise accentuated in the reports generated by the report module 134.

Report module 134 may generate reports 140 to provide to the user based on the matching and consolidation of information from the genomic database 119 performed by the matching and consolidation module 132. The report module 134 may provide listings of gene-mutations and therapies matched by the matching and consolidation module 132 with signature mutations accentuated in the listing. The report module may also comprise report text templates that may be populated with matching gene-mutation information, therapy information, clinical evidence information, and the like, obtained from the genomic database 119 in the matching entries. A combination of text template based reporting and tabular gene-mutation information may be included in the generated report 140 output by the report module 134 and which are presented to the user via the user interface 145.

For example, in response to the matching and consolidation module 132 identifying a matching entry in the genomics database 119 corresponding to a signature mutation, the report module 134 may utilize template text to generate a statement in the report 140 of the type “This tumor is characterized by the presence of [gene-mutation] . . . Clinical evidence demonstrates that the presence of this mutation is a strong predictor of response to treatment with [relevant therapy] as indicated by [actual values from evidence]. Investigational therapies may also be considered.” In this template text, the particular gene-mutation identified in the matching entry corresponding to the signature mutation may be inserted into the field “[gene-mutation]”, the highest scoring therapy, i.e. the relevant therapy, corresponding to that signature mutation as indicated in the matching entry may be inserted into the field “[relevant therapy]”, and values supportive of the gene-mutation being a signature mutation as determined by the evaluation performed by the biomarker grading ML computer model 155 may be used to populate the field(s) “[actual values from evidence].” Such textual statements may be included in the report 140 and may be highlighted or otherwise accentuated in the summary of the report 140 to distinguish this gene-mutation and therapy from the description of other mutations associated with treatment, resistance, prognosis, or diagnostic factors that may also be present in the same tumor. In addition, a visual cue or other highlighting or accentuation in a corresponding table below the summary to highlight the signature mutation and its therapy. Grading values may be presented in the table in association with the various gene-mutations matched, with the signature mutations having a corresponding grading value of IIII, strong driver gene-mutations indicated with a grading value of III, and the like.

The genomic database 119 mechanisms may be implemented by any conventional or other database or storage unit, may be local to or remote from server systems 110 and client systems 114, and may communicate via any appropriate communication medium (e.g., local area network (LAN), wide area network (WAN), Internet, hardwire, wireless link, Intranet, etc.). The client systems may present a graphical user (e.g., GUI, etc.) or other user interface 145 (e.g., command line prompts, menu screens, etc.) to solicit information from users pertaining to the desired documents and analysis, and may provide reports 140 including analysis results (e.g., case summaries, genes, gene variants, variant types, condition names, evidence, prognoses, diagnoses/diseases, cancer types, predispositions, mutations (e.g., somatic or germline), treatments or therapies, etc.). As noted above, of particular relevance to the illustrative embodiments, the reports 140 may include accentuated representations of signature mutations found in the driver gene-mutations identified by the molecular profile analysis module 131 through the matching performed by the matching and consolidation module 132.

Server systems 110 and client systems 114 may be implemented by any conventional or other computer systems preferably equipped with a display or monitor, a base (e.g., including at least one processor 115, one or more memories 135 and/or internal or external network interfaces or communications devices 125 (e.g., modem, network cards, etc.)), optional input devices (e.g., a keyboard, mouse or other input device), and any commercially available and custom software (e.g., server/communications software, molecular profile analysis module 131, matching and consolidation module 132, report module 134, browser/interface software, etc.). Moreover, the server systems 110 may be specifically configured with customized logic as discussed above for implementing an AI pipeline 150 comprising a plurality of trained ML computer models 152-155 specifically trained to perform the various operations detailed above, orchestrate the execution and interaction of these trained ML computer models in an automated manner, and generate genetic reports specifically identifying gradings and relevant therapies for gene-mutations, and especially signature mutations that are particularly accentuated in these reports 140 for ease of identification by a human user.

Alternatively, one or more client systems 114 may generate reports 140 when operating as a stand-alone unit. In a stand-alone mode of operation, the client system stores or has access to the data (e.g., corpus/corpora 118, clinical input data 105, genomic database 119, etc.), and includes the AI pipeline 150, molecular profile analysis module 131 and matching and consolidation module 132 to perform gene-mutation grading and relevant therapy identification based on scoring, molecule profiling analysis, and matching and consolidation of data with the genomic database 119 to generate reports 140. The graphical user (e.g., GUI, etc.) or other interface (e.g., command line prompts, menu screens, etc.) may solicit information from a corresponding user pertaining to the desired documents and analysis, and may provide reports 140 including analysis results.

Server 110 may include one or more modules or units to perform the various functions of present invention embodiments described herein. The various modules (e.g., molecular profile analysis module 131, and matching and consolidation module 132, report module 134, etc.), the natural language processing logic and resources 151, and the machine learning (ML) computer models 152-155 may be implemented by any combination of any quantity of software and/or hardware modules or units, and may reside within memory 135 of the server and/or client systems for execution by one or more processors 115.

Clinical input data 105 may comprise patient gene sequences, e.g., tumor sequences in a Variant Call Format (VCF), which may be analyzed by the molecular profile analysis module 131 to identify driver gene mutations. A listing of driver gene mutations may be provided to the molecular profile analysis module 131 and may be derived from any suitable source (e.g., literature, cancer databases such as The Cancer Genome Atlas, exome sequencing information, etc.). The listing of driver gene mutations may be curated (e.g., manually or in an automated manner) prior to providing to the molecular profile analysis module 131, and may be stored in any suitable database. Cancer cells may have thousands of mutations, and a patient may have different metastases with different mutations. Mutations in driver genes may be common or similar across these different metastases, and are targets for drug development and cancer treatment.

The matching and consolidation module 132 may comprise hierarchical matching techniques, such as described in commonly assigned and co-pending U.S. patent application Ser. No. 16/371,204 entitled “Extracting Related Medical Information From Different Data Sources for Automated Generation of Prognosis, Diagnosis, and Predisposition Information in Case Summary,” filed Apr. 1, 2019, which is hereby incorporated herein by reference in its entirety. The hierarchical matching technique associates genetic information obtained from a patient with curated literature from the corpus/corpora 118, which may be stored as tables or other structured data structures in genomic database 119, to provide prognostic, diagnostic, and predisposition information.

As described in the co-pending and commonly assigned U.S. patent application Ser. No. 16/371,204, in order to perform such a hierarchical matching technique, for a given patient's gene sequencing data, molecular profile analysis module 131 may analyze the gene sequencing data to obtain a list of the driver genes with pathogenic/vus mutations. For each driver gene mutation, matching and consolidation module 132 compares (e.g., using hierarchical matching) mutation data to the curated genomic knowledge in genomic database 119 obtained through the above AI pipeline 150 processes, to determine if there is a match. The matching and consolidation module 132 has a hierarchical progression starting from the smallest scope at a specific mutation progressing to a large scope. For example, if a match is not found at the specific mutation level, then the matching scope is gradually enlarged until a match is found or the system determines that no relevant entry is found. Matching and consolidation module 132 may also perform a cancer type progression, from specific/relevant cancers through parent/child relationships in cancer ontology, cancer categories (solid/hematological) and to the largest scope for any cancer. The hierarchical progression of this matching may be guided by one or more predetermined ontologies, such as ontologies for various types of cancer.

For example, the matching and consolidation module 132 may retrieve specific biomarkers from level 1 of an ontology, and may search the retrieved biomarker entries in the genomic database 119 to determine if there is a match with the patient sample. If a match is found, a result is returned. If a match is not found, the matching and consolidation module 132 moves up one level and retrieves biomarkers for a parent type of cancer, i.e. level 2, using the parent/child relationships represented in the ontology. For example, a parent relationship for a breast cancer category may include reproductive organ cancer. In level 2, parent biomarkers (and corresponding subcategories) are searched to determine if there is a match with the patient sample. If a match is found, a result is returned and if a match is not found, the matching and consolidation module 131 may move up one level of the ontology and retrieve biomarkers for broader categories of cancer, covering solid and blood based diseases, in level 3 of the ontology. This process may be repeated to thereby traverse levels of the ontology and retrieve biomarkers from the various levels and perform matching operations. In general, the system starts at a specific level, and traverses the ontology to progressively broader levels in order to determine a match. Whenever the system moves up a level, biomarkers within that level (and corresponding lower levels) may be evaluated (e.g., breast cancer may include all BRCA genes and variants; reproductive organ cancer may include breast, ovarian and testicular cancer, etc.; and solid cancer may include all types of solid cancer, etc.).

In addition, in accordance with the illustrative embodiments described above, the hierarchical matching technique of U.S. patent application Ser. No. 16/371,204 is augmented by the mechanisms of the illustrative embodiments directed to identify matching gene-mutations that represent various gradings based on patient response and, in particular, signature mutations identified via such a grading mechanism. That is, as part of the hierarchical matching technique, when matching biomarkers are found for a particular level of the ontology, the resulting information present in the genomics database 119 is retrieved and utilized to generate a report, which as discussed above may include accentuating or otherwise including conspicuous content and/or visual cues to identify signature mutations within the generated report 140.

The report 140 may comprise prognostic, diagnostic and/or predisposition information, as well as signature mutation, relevant therapy, and gene-mutation grading information generated by the mechanisms of the illustrative embodiments. The report 140 may be generated by there port module 134 and transmitted via the network 112 to client systems 114. An embodiment of the report 140 may provide only prognosis information, only diagnosis information, only predisposition information, or any combination thereof, along with the signature mutation information, relevant therapy information, and/or gene-mutation grading information depending on the desired implementation and depending on the particular types of information requested by a user via the user interface 145. The content of the report may include textual statements and one or more tables for organizing the various types of information, e.g., prognostic, diagnostic, or predisposition information. Signature mutation, corresponding relevant therapy information, and gene-mutation gradings may be presented in these tables with signature mutations accentuated or otherwise conspicuously identified so as to bring a human user's attention to the signature mutations more expeditiously.

For prognostic data, the report that is generated by the report module 134 may provide a prognosis relative to a disease. In the case of cancer, the report 140 may provide a prognosis based on the specific genetic mutation(s) identified within the patient's cancer. In some cases, the specific genetic mutation(s) may be described as affecting prognosis in the patient's tumor type. In this case, the report may include an example text stating “[mutation type] of gene A is a predictor of [value] prognosis in [cancer type]” generated by the report module 134. The extracted relationships from the corpus/corpora 118 of curated electronic documents may additionally quantify the prognosis as poor, good, controversial, intermediate value levels, or the like, and this “value” may be used to populate the [value] field of this text.

For diagnostic data, the report may link identified genetic mutations to diseases. For example, if a specific genetic mutation is identified, the matching and consolidation module 132 may perform analysis to determine whether the mutation is known to be associated with (considered a hallmark of), or diagnostic of, a specific cancer type. In this case, the report generated by the report module 134 may include an example text stating “[mutation] is a diagnostic marker for [cancer type]”.

For predisposition data, the report may include links between specific genetic mutations that have been associated with a predisposition to a disease, such as hereditary cancer syndromes, for example. Also, for the predisposition table, entries for somatic and germline gene sequencing data may be present. Generally, two scenarios may be considered. For the first scenario, a tumor-only sample does not distinguish a germline mutation from a somatic mutation. In this case, the following example text may be generated by the report module 134 and included in the report: “A pathogenic mutation in the [gene name] gene has been detected. Pathogenic germline mutations in [gene name] have been associated with hereditary cancer.”

For the second scenario, normal or no-tumor DNA and tumor DAN from the patient may be both provided and it may be determined whether a genetic mutation is present in germline DNA. For germline mutations, the report module 134 may provide an example text in the report of the type: “A pathogenic germline mutation in the [gene name] gene has been detected. Pathogenic germline mutations in [gene name] have been associated with hereditary cancer.” If mutations are found in more than one gene, the report module 134 may include example text of the following type in the report: “Pathogenic germline mutations in [gene name 1], [gene name 2] . . . and [gene name N] have been detected. Pathogenic germline mutations in these genes have been associated with hereditary cancer.

Thus, the report module 134 may include a variety of templates to provide diagnostic, prognostic, and predisposition data regarding a patient to a user via a report and user interface. In addition, as discussed previously, the report module 134 may further include text templates that specifically identify signature mutations. As noted above, an example of such text templates may be: “This tumor is characterized by the presence of [gene-mutation] . . . Clinical evidence demonstrates that the presence of this mutation is a strong predictor of response to treatment with [relevant therapy] as indicated by [actual values from evidence]. Investigational therapies may also be considered.” Any one or more of the example template textual statements may be included in the report 140 alone or in combination with tabular information listing gene-mutations and their corresponding information, including gradings of the gene-mutations, relevant therapies as identified through the scoring mechanisms previously described above, and visual cues indicating signature mutations conspicuously relative to other listed gene-mutations in the tabular information of the report.

In some illustrative embodiments, the report may additionally include information about whether or not the gene-mutation is pathogenic, whether or not the mutation is associated with resistance to a therapy (e.g., drug), a list of therapies associated with treatment of the gene-mutation(s), a list of clinical trials and locations associated with the gene-mutation(s), etc. If a therapy/treatment for the type of gene-mutation or cancer has been approved by a regulatory agency, the report module 134 may provide that information about the approval of the therapy/treatment in the report. In some illustrative embodiments, the report may include an annotated genetic sequence listing corresponding to the tumor, listing the specific gene-mutations as determined by the molecular profile analysis module 131, and associated knowledge regarding prognosis, diagnosis, predisposition, treatment options, signature mutations, gradings, and the like. The report may rank these in accordance with the grading generated by the mechanisms of the illustrative embodiment such that, for example, grade IIII gene-mutations and their relevant therapies may be ranked higher than other gene-mutations and therapies in the listing.

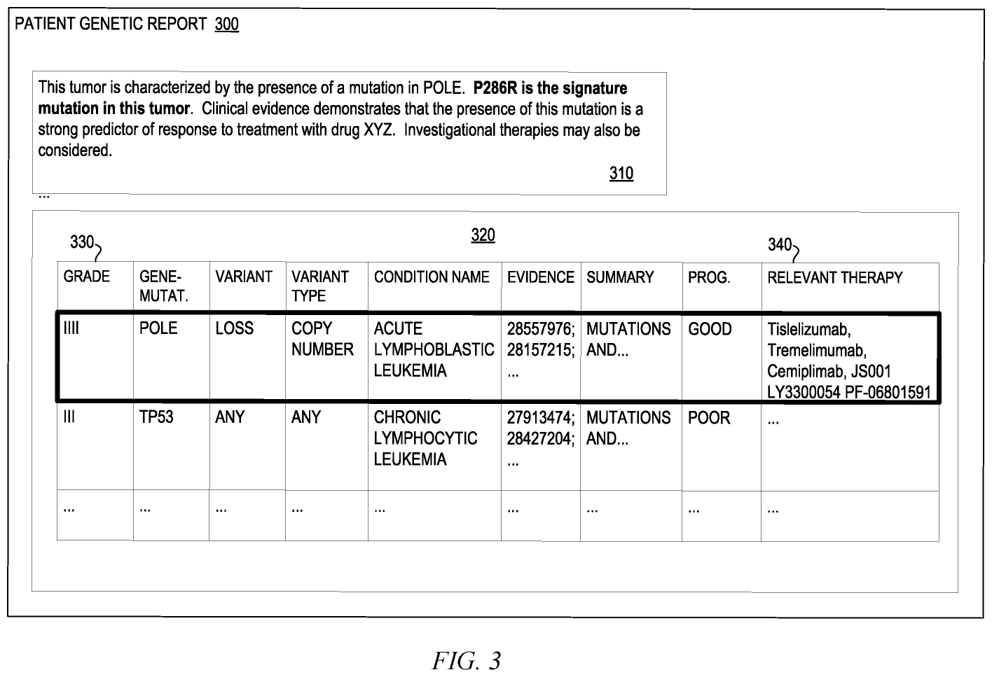

FIG. 3 is an example diagram of an example report that may be generated by a report module in accordance with one illustrative embodiment. It should be appreciated that the report 300 in FIG. 3 is specifically generated based on the identification of relationships between gene-mutations, therapies, and patient responses performed by the AI pipeline and its corresponding trained ML computer models as described previously, as well as the scoring of therapies and the grading of gene-mutations as described previously. In particular, the report 300 comprises signature mutation identification and emphasis in the report in accordance with the illustrative embodiments.

As shown in FIG. 3, the report 300 includes a first portion 310 comprising one or more textual descriptions generated by the report module 134 based on template text corresponding to the particular prognosis, diagnostic, predisposition, and/or signature mutation identification performed by the matching and consolidation module 132. In the depicted example, the first portion 310 comprises a textual statement that specifically identifies a signature mutation found through matching the patient's driver gene-mutations identified by the molecular profile analysis module 131 based on the patient's gene sequencing data, with entries in the genomic database 119. The content of the text is similar to the examples of template text provided previously.

The report 300 also includes a second portion 320 comprising tabular information listing gene-mutations matched with the patient's driver gene-mutations from the patient's gene sequencing data. As shown in FIG. 3, this second portion 320 may comprise entries having fields in which the particular gene-mutation name, variant, variant type, medical condition name corresponding to the gene-mutation, links to evidence describing the gene-mutation in the corpus/corpora, a summary description, and prognosis information are provided. In addition, in accordance with the mechanisms of the illustrative embodiments, the second portion 320 may include grading information 330 generated by the mechanisms of the illustrative embodiments to grade the gene-mutations with regard to patient responsiveness to the most relevant therapy for the gene-mutations, e.g., grades I-IIII. Moreover, the second portion 320 may specify the particular most relevant therapy 340 as indicated through the therapy scoring mechanism previously described above.

The listing of gene-mutations may be ranked relative to each other based on gradings 330 and/or scores associated with the relevant therapies. That is, in general there will be a single signature mutation associated with a tumor such that only one entry with a grading of IIII will be present in the listing. For other gradings, in which there may be multiple listings of gene-mutations, these may be ranked relative to each other based on the relative scores associated with their most relevant therapies. The signature mutation may be provided as a first entry in the tabular information so as to conspicuously identify the signature mutation relative to the other gene-mutations. Moreover, various visual cues, highlighting, or the like, may be applied to the representation of the signature mutation in the portions 310 and 320 of the report 300 to even further accentuate the signature mutation information relative to other information presented in the content of the report 300.

Thus, the illustrative embodiments provide an automated AI-pipeline for evaluating content of a corpus/corpora of curated genetic information to extract relationships between gene-mutations, therapies, and patient responses. The automated AI-pipeline mechanisms of the illustrative embodiments provide a plurality of trained ML computer models to perform classification of documents, entity extraction, relationship extraction, therapy scoring, and gene-mutation grading so as to identify signature mutations for medical conditions (e.g., cancer types, tumor types, etc.). This information is used to build a genomics database which is then used to match to a patient's gene sequencing data and generate a report regarding matching gene-mutations identified in the patient's gene sequencing data. This process is performed in an automated manner without any human intervention. The only human intervention is with regard to submitting a request for analysis of a patient's gene sequencing data and viewing the report output generated via the user interface. Because the reports generated by the automated AI mechanisms of the illustrative embodiments conspicuously identify signature mutations relative to the voluminous amount of gene-mutation information present in a patient's gene sequencing data, the attention of human subject matter experts (SMEs) to signature mutations whose associated therapies can cause the patient to have a complete response is made more expeditious and reduces the likelihood that the human SME will miss such signature mutations in the volume of genetic information.

As discussed upon above with regard to FIG. 1, the present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

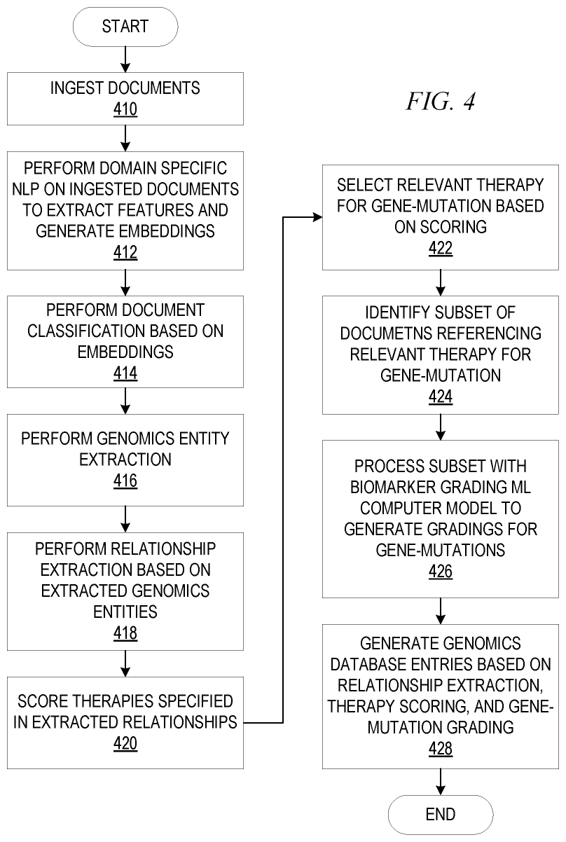

FIG. 4 is a flowchart outlining an example operation for automated extraction of gene-mutation relationships from a corpus of curated electronic documents, scoring therapies associated with the gene-mutation relationships, and grading gene-mutations in accordance with one illustrative embodiment. The operation outlined in FIG. 4 may be performed, for example, by the AI pipeline mechanism of the illustrative embodiments as described previously with regard to FIG. 1. It should be appreciated that the operations described in steps of the flowchart of FIG. 4 are performed by specifically configured and trained machine learning computer models of the AI pipeline in an automated manner.

As shown in FIG. 4, the operation starts with ingestion of a curated corpus/corpora of electronic documents to generate in-memory data representations of the content of the electronic documents (step 410). In some illustrative embodiments, the electronic documents may represent genomics domain specific electronic documents and the ingestion may be limited to particular select portions of the content, such as Abstracts, Findings, Conclusions, etc. where it is most likely that relationships between gene-mutations, therapies, and patient responses may be documented. The ingestion of the documents may include the execution of natural language processing logic and resources to the content of these documents to extract features and encode the features as vector representations of the content, such as using embedding tools as previously described above (step 412). The embedding representation of the electronic documents is input to a document classification ML model which classifies the documents according to a predetermined set of classes, such as “Functional”, “Clinical”, “Predisposition”, “Preclinical”, “Resistance”, and “Other” (step 414). Entity extraction is performed on documents of selected classes (step 416) with regard to genomics entities, such as genes, mutations, therapies, patient responses, etc. The semantic context of the extracted entities in the documents is evaluated to extract relationships between gene-mutations, therapies, and corresponding patient responses as documented in the ingested documents (step 418). For each gene-mutation, the therapies associated with the gene-mutation are scored to identify the most relevant therapies for the particular gene-mutation, where the most relevant therapy is the therapy providing the most positive outcome in patient response (step 420). Based on the therapy scoring, a relevant therapy for each gene-mutation is identified (step 422).

For each of the gene-mutations, the corpus/corpora of electronic documents are then processed to identify a subset of documents referencing the relevant therapy (step 424). This subset of documents is then processed by a biomarker grading ML computer model to grade the gene-mutation with regard to the most relevant therapy based on evidence present in the subset of documents (step 426). Based on the results of the relationship extraction, therapy scoring, and gene-mutation grading, entries in a genomic database are generated (step 428). The operation then terminates.

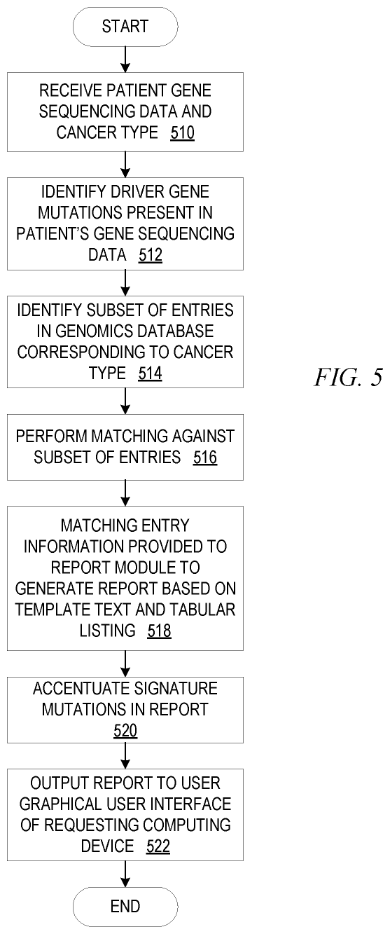

FIG. 5 is a flowchart outlining an example operation for performing a report generation based on matching of driver gene-mutations in a patient's gene sequencing data with entries in a genomic database in accordance with one illustrative embodiment. The operation outlined in FIG. 5 assumes the process for building the genomics database based on relationship extraction, therapy scoring, and gene-mutation grading has been performed such that entries in the genomics database store the grading and most relevant therapy information for each gene-mutation. The operations outlined in FIG. 5 may be performed, for example, by a molecular profile analysis module, a matching and consolidation module, and report module, such as shown and discussed with reference to FIG. 1 above.

As shown in FIG. 5, the operation starts by receiving gene sequencing data and cancer type information for a patient, such as in a request from a client computing device requesting a genetic report for the patient (step 510). The gene sequencing data and cancer type information is processed by the molecular profile analysis module to identify driver gene mutations present in the patient's gene sequencing data (step 512). The driver gene mutation information and cancer type information are used by a matching and consolidation module to identify a subset of entries in a genomics database corresponding to the cancer type (step 514). The subset of entries are then used as a basis for matching against the identified driver gene-mutations in the patient's gene sequencing data (step 516). The matching entries are provided to a report module which generates a report based on template text and/or tabular information obtained from the matching entries (step 518). Signature mutations, if any, that are matched by the matching and consolidation module, as indicated by the grading information, are highlighted in the generated report (step 520). The report is then provided to a user interface of the requesting client computing device for output to a user for review (step 522). The operation then terminates.

Thus, the illustrative embodiments provide improved computer tools employing automated AI mechanisms to automatically identify signature mutations and identify signature mutations in patient gene sequencing data such that corresponding therapies may be accentuated in reports generated for patients. It is apparent from the above that the improved computing tools of the illustrative embodiments may be part of a variety of different data processing environments including stand-alone devices or distributed data processing environments. FIG. 1 provides an example illustrative embodiment in which the improved computing tools are implemented using server and/or client computing devices along with corresponding data networks and the like. The individual computing devices, e.g., server computing system and client computing system, may take many different forms depending on the desired computing technology for the particular implementation.

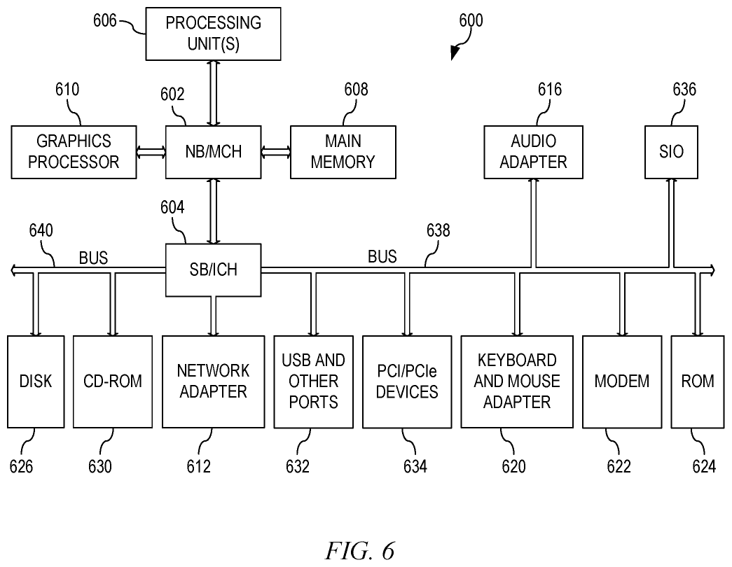

FIG. 6 is a block diagram of just one example data processing system or computing device in which aspects of the illustrative embodiments may be implemented. Data processing system 600 is an example of a computer, such as server 110 or client 114 in FIG. 1, in which computer usable code or instructions implementing the processes and aspects of the illustrative embodiments of the present invention may be located and/or executed so as to achieve the operation, output, and external effects of the illustrative embodiments as described herein.

In the depicted example, data processing system 600 employs a hub architecture including north bridge and memory controller hub (NB/MCH) 602 and south bridge and input/output (I/O) controller hub (SB/ICH) 604. Processing unit 606, main memory 608, and graphics processor 610 are connected to NB/MCH 602. Graphics processor 610 may be connected to NB/MCH 602 through an accelerated graphics port (AGP).

In the depicted example, local area network (LAN) adapter 612 connects to SB/ICH 604. Audio adapter 616, keyboard and mouse adapter 620, modem 622, read only memory (ROM) 624, hard disk drive (HDD) 626, CD-ROM drive 630, universal serial bus (USB) ports and other communication ports 632, and PCI/PCIe devices 634 connect to SB/ICH 604 through bus 638 and bus 640. PCI/PCIe devices may include, for example, Ethernet adapters, add-in cards, and PC cards for notebook computers. PCI uses a card bus controller, while PCIe does not. ROM 624 may be, for example, a flash basic input/output system (BIOS).

HDD 626 and CD-ROM drive 630 connect to SB/ICH 604 through bus 640. HDD 626 and CD-ROM drive 630 may use, for example, an integrated drive electronics (IDE) or serial advanced technology attachment (SATA) interface. Super I/O (SIO) device 636 may be connected to SB/ICH 604.

An operating system runs on processing unit 606. The operating system coordinates and provides control of various components within the data processing system 600 in FIG. 6. As a client, the operating system may be a commercially available operating system such as Microsoft® Windows 10®. An object-oriented programming system, such as the Java™ programming system, may run in conjunction with the operating system and provides calls to the operating system from Java™ programs or applications executing on data processing system 600.

As a server, data processing system 600 may be, for example, an IBM eServer™ System p® computer system, Power™ processor based computer system, or the like, running the Advanced Interactive Executive (AIX®) operating system or the LINUX® operating system. Data processing system 600 may be a symmetric multiprocessor (SMP) system including a plurality of processors in processing unit 606. Alternatively, a single processor system may be employed.

Instructions for the operating system, the object-oriented programming system, and applications or programs are located on storage devices, such as HDD 626, and may be loaded into main memory 608 for execution by processing unit 606. The processes for illustrative embodiments of the present invention may be performed by processing unit 606 using computer usable program code, which may be located in a memory such as, for example, main memory 608, ROM 624, or in one or more peripheral devices 626 and 630, for example.

A bus system, such as bus 638 or bus 640 as shown in FIG. 6, may be comprised of one or more buses. Of course, the bus system may be implemented using any type of communication fabric or architecture that provides for a transfer of data between different components or devices attached to the fabric or architecture. A communication unit, such as modem 622 or network adapter 612 of FIG. 6, may include one or more devices used to transmit and receive data. A memory may be, for example, main memory 608, ROM 624, or a cache such as found in NB/MCH 602 in FIG. 6.

As mentioned above, in some illustrative embodiments the mechanisms of the illustrative embodiments may be implemented as application specific hardware, firmware, or the like, application software stored in a storage device, such as HDD 626 and loaded into memory, such as main memory 608, for executed by one or more hardware processors, such as processing unit 606, or the like. As such, the computing device shown in FIG. 6 becomes specifically configured to implement the mechanisms of the illustrative embodiments and specifically configured to perform the operations and generate the outputs described herein with regard to the AI pipeline, matching and consolidation module, molecular profile analysis module, report module, and the like.

Those of ordinary skill in the art will appreciate that the hardware in FIGS. 1 and 6 may vary depending on the implementation. Other internal hardware or peripheral devices, such as flash memory, equivalent non-volatile memory, or optical disk drives and the like, may be used in addition to or in place of the hardware depicted in FIGS. 1 and 6. Also, the processes of the illustrative embodiments may be applied to a multiprocessor data processing system, other than the SMP system mentioned previously, without departing from the spirit and scope of the present invention.

Moreover, the data processing system 600 may take the form of any of a number of different data processing systems including client computing devices, server computing devices, a tablet computer, laptop computer, telephone or other communication device, a personal digital assistant (PDA), or the like. In some illustrative examples, data processing system 600 may be a portable computing device that is configured with flash memory to provide non-volatile memory for storing operating system files and/or user-generated data, for example. Essentially, data processing system 600 may be any known or later developed data processing system without architectural limitation.

It should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc. A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or I/O devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Modems, cable modems and Ethernet cards are just a few of the currently available types of network adapters for wired communications. Wireless communication based network adapters may also be utilized including, but not limited to, 802.11 a/b/g/n wireless communication adapters, Bluetooth wireless adapters, and the like. Any known or later developed network adapters are intended to be within the spirit and scope of the present invention.

The description of the present invention has been presented for purposes of illustration and description, and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, in a data processing system comprising at least one processor and at least one memory, the at least one memory comprising instructions executed by the at least one processor to configure the at least one processor to implement a genomics artificial intelligence (AI) pipeline comprising a plurality of trained machine learning computer models, wherein the method comprises:
processing, by at least one trained first machine learning (ML) computer model of the genomics AI pipeline, a corpus of electronic documents to extract genomics entities from content of the electronic documents;
processing, by at least one trained second ML computer model of the genomics AI pipeline, the extracted genomics entities to generate one or more relationships between the extracted genomics entities;
processing, by at least one trained third ML computer model of the genomics AI pipeline, the one or more relationships to grade biomarkers specified in the one or more relationships based on a predetermined grading scheme to thereby generate gradings for each of the one or more relationships;
storing, by the genomics AI pipeline, the one or more relationships in association with corresponding gradings of the one or more relationships in a genomics database;
processing, by a matching and consolidation module associated with the genomics AI pipeline, a patient gene sequencing data structure based on the genomics database to identify a signature mutation in the patient gene sequencing data structure by matching a gene mutation in the patient gene sequencing data structure to an entry in the genomics database corresponding to the signature mutation; and
generating, by a report module associated with the genomics AI pipeline, a report output identifying the signature mutation present in the patient gene sequencing data structure.

2. The method of claim 1, wherein the at least one trained first ML computer model comprises a document classification ML computer model that is trained to classify electronic documents in the corpus of electronic documents as to types of clinical studies documented in content of the electronic documents, to thereby generate one or more subsets of electronic documents in the corpus of electronic documents, each subset corresponding to a different type of clinical study, and wherein the method further comprises executing the document classification ML computer model on electronic documents of the corpus of electronic documents and filtering out documents from further processing by the genomics AI pipeline, that have a predefined type.

3. The method of claim 2, wherein the at least one trained first ML computer model comprises a genomics entity extraction ML computer model that is configured, for each subset of electronic documents in the one or more subsets of electronic documents, to extract a subset of types of genomics entities based on a class of the subset of electronic documents.

4. The method of claim 1, wherein the at least one trained second ML computer models comprise genomic relationship scoring logic that scores each relationship of the one or more relationships based on features specifying a clinical efficacy of a therapy associated with a genetic mutation specified in the relationship.

5. The method of claim 4, wherein the scoring logic assigns different scores to different types of patient response to a corresponding therapy, and wherein, for each relationship in the one or more relationships, scores for instances in content of electronic documents of the corpus, of a gene mutation-therapy pair specified in the relationship, are accumulated across the instances to generate a score for the relationship, and wherein different therapies for a same gene mutation are ranked relative to each other based on corresponding accumulated scores for corresponding gene mutation-therapy pairs.

6. The method of claim 1, wherein processing the one or more relationships to grade biomarkers comprises, for each relationship in the one or more relationships, classifying, by the at least one trained third ML computer model, the relationship into a corresponding grade of the predetermined grading scheme, wherein the grading scheme comprises:
a first grade indicating that no specific biomarker preclinical data or response is expected;
a second grade indicating that biomarker responses have not been reported;
a third grade indicating strong biomarker and strong response or lasting response in a plurality of patients; and
a fourth grade indicating a signature mutation with complete remission or lasting partial response in patients.

7. The method of claim 1, wherein processing the patient gene sequencing data structure based on the genomics database to identify the signature mutation comprises:
receiving, from a molecular profile analysis module, the gene sequencing data, wherein the gene sequencing data comprises driving gene-mutations present in a gene sequence for a tumor of the patient;
receiving, from the molecular profile analysis module, an indicator of a type of cancer associated with the patient;
performing a lookup operation in the genomics database a subset of entries corresponding to the type of cancer; and
performing, by the matching and consolidation module, a lookup operation on the subset of entries corresponding to the cancer type, for each driving gene-mutation in the gene sequencing data, to find a corresponding matching entry in the subset of entries, if any.

8. The method of claim 1, wherein generating the report output identifying the signature mutation present in the patient gene sequencing data structure further comprises accentuating the signature mutation in a display of the patient's genetic report and outputting a recommendation of a corresponding therapy based on the signature mutation, as indicated by the entry in the genomics database corresponding to the signature mutation.

9. The method of claim 1, wherein the genomics entities comprise gene mutations, therapies, medical conditions, and indicators of clinical efficacy of the therapies, and wherein the genomics entities are extracted from electronic documents of the corpus of electronic documents by executing natural language processing computer operations on content of the electronic documents.

10. The method of claim 1, wherein the genomics entities and relationships between genomics entities are associated with one or more of solid tumors or hematology.

11. A computer program product comprising a computer readable storage medium having a computer readable program stored therein, wherein the computer readable program, when executed by a data processing system, causes the data processing system to implement a genomics artificial intelligence (AI) pipeline comprising a plurality of trained machine learning computer models that operate to:
process, by at least one trained first machine learning (ML) computer model of the genomics AI pipeline, a corpus of electronic documents to extract genomics entities from content of the electronic documents;
process, by at least one trained second ML computer model of the genomics AI pipeline, the extracted genomics entities to generate one or more relationships between the extracted genomics entities;
process, by at least one trained third ML computer model of the genomics AI pipeline, the one or more relationships to grade biomarkers specified in the one or more relationships based on a predetermined grading scheme to thereby generate gradings for each of the one or more relationships;
store, by the genomics AI pipeline, the one or more relationships in association with corresponding gradings of the one or more relationships in a genomics database;
process, by a matching and consolidation module associated with the genomics AI pipeline, a patient gene sequencing data structure based on the genomics database to identify a signature mutation in the patient gene sequencing data structure by matching a gene mutation in the patient gene sequencing data structure to an entry in the genomics database corresponding to the signature mutation; and
generate, by a report module associated with the genomics AI pipeline, a report output identifying the signature mutation present in the patient gene sequencing data structure.

12. The computer program product of claim 11, wherein the at least one trained first ML computer model comprises a document classification ML computer model that is trained to classify electronic documents in the corpus of electronic documents as to types of clinical studies documented in content of the electronic documents, to thereby generate one or more subsets of electronic documents in the corpus of electronic documents, each subset corresponding to a different type of clinical study, and wherein the method further comprises executing the document classification ML computer model on electronic documents of the corpus of electronic documents and filtering out documents from further processing by the genomics AI pipeline, that have a predefined type.

13. The computer program product of claim 12, wherein the at least one trained first ML computer model comprises a genomics entity extraction ML computer model that is configured, for each subset of electronic documents in the one or more subsets of electronic documents, to extract a subset of types of genomics entities based on a class of the subset of electronic documents.

14. The computer program product of claim 11, wherein the at least one trained second ML computer models comprise genomic relationship scoring logic that scores each relationship of the one or more relationships based on features specifying a clinical efficacy of a therapy associated with a genetic mutation specified in the relationship.

15. The computer program product of claim 14, wherein the scoring logic assigns different scores to different types of patient response to a corresponding therapy, and wherein, for each relationship in the one or more relationships, scores for instances in content of electronic documents of the corpus, of a gene mutation-therapy pair specified in the relationship, are accumulated across the instances to generate a score for the relationship, and wherein different therapies for a same gene mutation are ranked relative to each other based on corresponding accumulated scores for corresponding gene mutation-therapy pairs.

16. The computer program product of claim 11, wherein processing the one or more relationships to grade biomarkers comprises, for each relationship in the one or more relationships, classifying, by the at least one trained third ML computer model, the relationship into a corresponding grade of the predetermined grading scheme, wherein the grading scheme comprises:
a first grade indicating that no specific biomarker preclinical data or response is expected;
a second grade indicating that biomarker responses have not been reported;
a third grade indicating strong biomarker and strong response or lasting response in a plurality of patients; and
a fourth grade indicating a signature mutation with complete remission or lasting partial response in patients.

17. The computer program product of claim 11, wherein processing the patient gene sequencing data structure based on the genomics database to identify the signature mutation comprises:
receiving, from a molecular profile analysis module, the gene sequencing data, wherein the gene sequencing data comprises driving gene-mutations present in a gene sequence for a tumor of the patient;
receiving, from the molecular profile analysis module, an indicator of a type of cancer associated with the patient;
performing a lookup operation in the genomics database a subset of entries corresponding to the type of cancer; and
performing, by the matching and consolidation module, a lookup operation on the subset of entries corresponding to the cancer type, for each driving gene-mutation in the gene sequencing data, to find a corresponding matching entry in the subset of entries, if any.

18. The computer program product of claim 11, wherein generating the report output identifying the signature mutation present in the patient gene sequencing data structure further comprises accentuating the signature mutation in a display of the patient's genetic report and outputting a recommendation of a corresponding therapy based on the signature mutation, as indicated by the entry in the genomics database corresponding to the signature mutation.

19. The computer program product of claim 11, wherein the genomics entities comprise gene mutations, therapies, medical conditions, and indicators of clinical efficacy of the therapies, and wherein the genomics entities are extracted from electronic documents of the corpus of electronic documents by executing natural language processing computer operations on content of the electronic documents.

20. An apparatus comprising:
a processor; and
a memory coupled to the processor, wherein the memory comprises instructions which, when executed by the processor, cause the processor to implement a genomics artificial intelligence (AI) pipeline comprising a plurality of trained machine learning computer models that operate to:
process, by at least one trained first machine learning (ML) computer model of the genomics AI pipeline, a corpus of electronic documents to extract genomics entities from content of the electronic documents;
process, by at least one trained second ML computer model of the genomics AI pipeline, the extracted genomics entities to generate one or more relationships between the extracted genomics entities;
process, by at least one trained third ML computer model of the genomics AI pipeline, the one or more relationships to grade biomarkers specified in the one or more relationships based on a predetermined grading scheme to thereby generate gradings for each of the one or more relationships;
store, by the genomics AI pipeline, the one or more relationships in association with corresponding gradings of the one or more relationships in a genomics database;
process, by a matching and consolidation module associated with the genomics AI pipeline, a patient gene sequencing data structure based on the genomics database to identify a signature mutation in the patient gene sequencing data structure by matching a gene mutation in the patient gene sequencing data structure to an entry in the genomics database corresponding to the signature mutation; and
generate, by a report module associated with the genomics AI pipeline, a report output identifying the signature mutation present in the patient gene sequencing data structure.
